(19) United States
(12) Patent Application Publication
(10) Pub. No.: US 2008/0300326 A1
(43) Pub. Date: Dec. 4, 2008

(54) Processing Residue Gas of a Fischer-Tropsch Process

(75) Inventors: Martine Schneider, Versailles (FR); Guillaume De Souza, Issy Les Moulineaux (FR); Paul Wentink, Feucherolles (FR)

Correspondence Address: AIR LIQUIDE;Intellectual Property, 2700 POST OAK BOULEVARD, SUITE 1800, HOUSTON, TX, 77056, US

(73) Assignee: L'Air Liquide Societe Anonyme Pour L'Etude Et L'Ex ploitation Des Procedes Georges Claude, Paris (FR)

(21) Appl. No.: 11/996,847

(22) PCT Filed: Jul. 26, 2006
(86) PCT No.: PCT/FR2006/001824

§ 371 Date: Jul. 28, 2008

(30) Foreign Application Priority Data
Jul. 28, 2005 (FR) 0552354

(51) Int. Cl. C07C 27/00

(52) U.S. Cl. 518/700

(57) ABSTRACT

The invention concerns a method for converting hydrocarbon-containing gases into hydrocarbon-containing liquids wherein the Fischer-Tropsch process is implemented, said Fischer-Tropsch process producing hydrocarbon-containing liquids and a residue gas comprising at least hydrogen, carbon monoxide, carbon dioxide and hydrocarbons having a carbon number not more than 6, wherein the residue gas is subjected to a separation method producing: at least one gas stream comprising for the major part hydrogen; at least one stream comprising for the major part methane; at least one gas stream comprising for the major part inerts (carbon dioxide, nitrogen, argon) and hydrocarbons having a carbon number not less than 2.

The present invention relates to a novel method for converting hydrocarbon gases to liquid hydrocarbons using one of the known processes for generating synthesis gas, and also the Fischer-Tropsch process, and particularly a specific step for treating the offgas issuing from the Fischer-Tropsch process.

It is known how to convert basic gaseous or solid hydrocarbon compounds to liquid hydrocarbons utilized in the petrochemical industry, in refineries, or in the transport sector. In fact, some large natural gas reservoirs are located in remote areas distant from any consumer areas; they can accordingly be employed by installing “gas to liquid” (GtL) conversion plants on a site close to these natural gas sources. Gas to liquid conversion allows easier transport of the hydrocarbons. This type of GtL conversion commonly involves converting the basic gaseous or solid hydrocarbon compounds to a synthesis gas mainly comprising H₂ and CO (by partial oxidation using an oxidizing gas and/or reaction with water vapour and/or CO₂), followed by the processing of this synthesis gas by the Fischer-Tropsch process to obtain a product which, after condensation, yields the desired liquid hydrocarbons. An offgas is produced during this condensation. This offgas contains low molecular weight hydrocarbons and unreacted gases. In consequence, it is generally used as fuel in one of the processes of the GtL unit, for example, in a gas turbine or a combustion chamber associated with a steam turbine, or in a flash turbine associated with a compressor of the GtL unit. However, the quantity of offgas to be burned often largely exceeds the fuel demand of the GtL unit. Moreover, the offgas also comprises CO₂, which decreases the hydrocarbon combustion efficiency and is salted out in the atmosphere, which is contrary to compliance with environmental standards. Finally, the offgas generally comprises unconverted quantities of H₂ and CO: it is therefore uneconomical to burn them.

WO 02/38699 describes a method for converting hydrocarbon gases to liquid hydrocarbons using the Fischer-Tropsch process, in which the offgas of this Fischer-Tropsch process is condensed to remove the hydrocarbon compounds containing more than 3 carbon atoms, and the gas resulting from this condensation is processed in order to produce: a stream comprising higher CO and H₂ concentrations, a CH₄ enriched stream, and a stream mainly comprising CO₂.

WO 2004/092306 describes a method for converting hydrocarbon gases to liquid hydrocarbons using the Fischer-Tropsch process, in which the offgas from this Fischer-Tropsch process is processed by a PSA separation unit in order to produce: a stream enriched with CH₄, CO and H₂, a CO₂ enriched stream, and a stream comprising hydrocarbons with more than two carbon atoms.

It is the object of the present invention to propose a method for converting hydrocarbon gases to liquid hydrocarbons using the Fischer-Tropsch process, in which the offgas from this Fischer-Tropsch process is processed in order to utilize the various components of this gas.

The invention permits the re-use, in the GtL process, of the compounds present in the offgas. The invention has the major advantage of distributing the various compounds of the offgas into several gas streams usable in various steps of the general method for converting hydrocarbon gases to liquid hydrocarbons, particularly for high conversion Fischer-Tropsch processes or for Fischer-Tropsch processes using a synthesis gas with a low H₂/CO ratio.

For this purpose, the invention relates to a method for converting hydrocarbon gases to liquid hydrocarbons using the Fischer-Tropsch process, the said Fischer-Tropsch process producing liquid hydrocarbons and an offgas comprising at least hydrogen, carbon monoxide, carbon dioxide and hydrocarbons with a maximum of 6 carbon atoms, in which the offgas is subjected to a separation process producing:

at least one gas stream mainly comprising hydrogen and having a lower CO concentration than the offgas,

at least one gas stream mainly comprising methane,

at least one gas stream mainly comprising:
inerts selected from carbon dioxide, nitrogen and/or argon, and
hydrocarbons with at least 2 carbon atoms.

The invention relates to any type of method for converting hydrocarbon gases to liquid hydrocarbons using the Fischer-Tropsch process. These hydrocarbon gases generally issue from a reaction for producing a hydrocarbon synthesis gas (for example, by partial oxidation using an oxidizing gas and water vapour). This synthesis gas comprises hydrogen and CO. It commonly issues from a unit for preparing a synthesis gas from natural gas or an associated gas or coal. According to the inventive method, this synthesis gas is subjected to a Fischer-Tropsch reaction by contact with a catalyst promoting this reaction.

During the Fischer-Tropsch reaction, the hydrogen and CO are converted to hydrocarbon compounds with variable chain lengths by the following reaction:

CO+(1+m/2n)H₂→(1/n)CₙHₘ+H₂0

CO₂ is also produced during this reaction; for example, by the following side reactions:

$$CO + {H}_{2} 0 \to {CO}_{2} + {H}_{2}$$

$$2 CO \to {CO}_{2} + C$$

At the outlet of the reactor using the Fischer-Tropsch process, the temperature of the products is generally lowered from a temperature of around 130° C., preferably to a temperature of around 90 to 60° C., so that, on the one hand, a condensate is obtained, mainly composed of water and liquid hydrocarbons with more than 4 carbon atoms, and, on the other, an offgas comprising at least hydrogen, carbon monoxide, hydrocarbons with no more than 6 carbon atoms, carbon dioxide and also generally comprising nitrogen.

The present invention relates to the processing of this offgas obtained. According to the inventive method, this offgas is subjected to a separation process producing:

at least one gas stream mainly comprising hydrogen and having a lower CO concentration than the offgas,

at least one gas stream mainly comprising methane,

at least one gas stream mainly comprising:
inerts selected from carbon dioxide, nitrogen and/or argon, and
hydrocarbons with at least 2 carbon atoms.

The expressions below have the following meaning according to the invention:

“gas stream mainly comprising a compound” means a gas stream of which the concentration by volume of this compound is higher than the concentration by volume of each of the other compounds making up this gas stream;

“gas stream mainly comprising inerts selected from carbon dioxide, nitrogen and argon, and hydrocarbons with at least 2 carbon atoms” means a gas stream of which the concentration by volume of these inerts and these hydrocarbons is higher than the concentration by volume of each of the other compounds making up this gas stream.

According to the invention, the first stream mainly comprising hydrogen generally has a hydrogen concentration of at least 70% by volume, preferably at least 90%. It generally comprises no more than 10% of the CO initially present in the offgas processed. Thus the CO concentration in the first stream is lower than 5% by volume, preferably lower than 2% by volume. This first stream commonly has a pressure of at least 1.6. 10⁶ Pa. This first stream may also comprise other compounds such as nitrogen, helium, argon and, optionally, carbon monoxide and methane.

The second stream mainly comprising methane generally has a methane concentration of at least 40% by volume, preferably at least 60%. It generally comprises at least 30% of the CO initially present in the offgas processed. Thus, the CO concentration of the second stream is between 5 and 25% by volume. This second stream commonly has a pressure of between 2.10⁵ and 1.6.10⁶ Pa. This second stream may also comprise other compounds such as nitrogen, helium, argon, hydrogen and carbon monoxide and dioxide.

The third stream mainly comprising inerts (carbon dioxide, nitrogen, argon) and hydrocarbons with at least 2 carbon atoms, generally has a carbon dioxide recovery of at least 50% and preferably at least 80%, and a recovery of hydrocarbons with at least 2 carbon atoms of at least 30%, preferably at least 80%. It generally comprises at least 50% of the CO initially present in the offgas. This third stream commonly has a pressure of no more than 8.10⁵ Pa. This third stream may also comprise minority quantities of other compounds.

According to the invention, the separation method for processing the offgas is preferably a pressure swing adsorption or PSA method. Depending on the various pressure cycles, the PSA separation method is suitable for obtaining in succession:

the first gas stream mainly comprising hydrogen, then

the second gas stream mainly comprising methane, then,

the third gas stream mainly comprising:
inerts (carbon dioxide, nitrogen, argon) and
hydrocarbons with at least 2 carbon atoms.
   Preferably, each adsorber of the PSA separation unit is composed of at least three adsorbent beds, and preferably at least four:

the first being composed of alumina,

the second being composed of carbon molecular sieves or silicalite,

the third being composed of activated carbon,

the fourth bed being optional and composed of zeolite.

The order of the beds indicated corresponds to the offgas flow direction in the adsorber.

The alumina is suitable for removing the water present in the offgas and the hydrocarbon compounds with five or more carbon atoms. On the contrary, the alumina allows any H₂, CO, CH₄, CO₂ and N₂ present in the offgas to pass through. Carbon molecular sieves and silicalites are suitable for adsorbing the carbon dioxide, and even partially methane. Preferably, the carbon molecular sieves have average pore sizes of between 2.8 and 5 Å and even more preferably, of between 3 and 3.8 Å. Similarly, it is preferable to use silicalites with a Si/Al molecular ratio of at least 3, such as in Hisiv silicalite 3000® from UOP. The activated carbon is suitable for adsorbing methane and partially nitrogen and carbon monoxide. The zeolite is suitable for adsorbing nitrogen, argon and carbon monoxide.

According to a variant of the invention, each adsorber of the PSA separation unit may also comprise a bed composed of silica gel placed between the first and the second bed. This bed is designed to protect the upper beds from the hydrocarbon compounds with three or more carbon atoms. Preferably, the silica gel used has an alumina (Al₂O₃) concentration of less than 1% by weight.

In practice, the stream mainly comprising hydrogen is obtained during the PSA production phase. The second stream mainly comprising methane is obtained during the decompression phase. The third stream mainly comprising inerts (carbon dioxide, nitrogen, argon) and hydrocarbons with at least 2 carbon atoms, is obtained at the end of the decompression phase of the PSA cycle and/or at the end of the low pressure elution step.

According to a first variant, to obtain the stream mainly comprising methane, each adsorber can be divided into two semi-adsorbers in series, the first semi-adsorber comprising the alumina bed, optionally the silica gel bed if any, and a fraction of the carbon molecular sieve or silicalite bed. The second semi-adsorber comprises the remaining fraction of the carbon molecular sieve or silicalite bed and of the activated carbon and zeolite beds. The stream mainly comprising methane is accordingly obtained at the outlet of the first semi-adsorber during the decompression phase of one or both semi-adsorbers.

According to a second variant, it is also possible to tap off the stream mainly comprising methane by a withdrawal means placed in the adsorber. Preferably, the withdrawal means is placed at the level of the second half of the bed composed of carbon molecular sieves or silicalite, in the flow direction of the offgas issuing from the Fischer-Tropsch process in the adsorber.

The present invention further relates to the use which can be made of the three gas streams issuing from the processing of the offgas. Thus, at least part of the gas stream mainly comprising hydrogen can be used as reagent gas in the Fischer-Tropsch process. At least part of the gas stream mainly comprising hydrogen can also be used in hydrocracking processes. These hydrocracking processes are frequently used on refinery sites close to the GtL units. This use is feasible if the gas stream mainly comprising hydrogen comprises less than 100 ppm of CO. If the CO concentration is too high, the stream mainly comprising hydrogen is preferably recycled as reagent in the Fischer-Tropsch process. At least part of the gas stream mainly comprising hydrogen can also be used to recover heat or work via a turbine. Finally, part of this stream can be sent to a fuel network.

Furthermore, at least part of the gas stream mainly comprising methane can be used as reagent gas in the generation of synthesis gas, for example as a reagent in a synthesis gas production process. This synthesis gas generation corresponds to the step prior to the implementation of the Fischer-Tropsch process.

At least part of the gas stream mainly comprising methane can also be used as reagent in a steam methane reforming or SMR process. At least part of the gas stream mainly comprising methane can be used in a CO₂ separation unit (amine scrubbing, for example) to sequester the CO₂.

Finally, part of the gas stream mainly comprising inerts (carbon dioxide, nitrogen, argon) and hydrocarbons with at least 2 carbon atoms can be used as fuel.

Prior to all the subsequent uses which can be made of the streams produced by the separation unit, the said streams can be heated by heat exchange with the offgas or a synthesis gas. The latter may be the synthesis gas used for the implementation of the Fischer-Tropsch process.

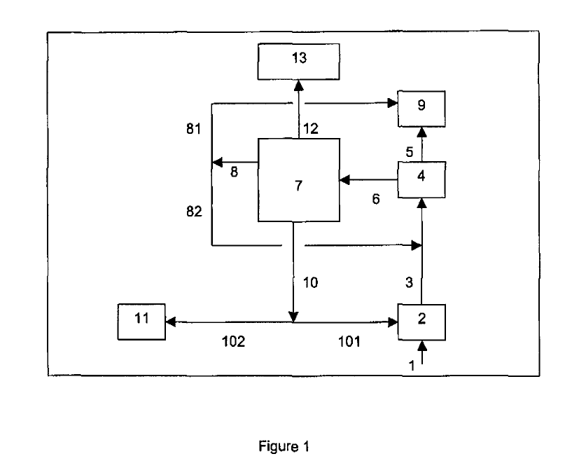

FIG. 1 shows the method according to the invention. Gaseous hydrocarbons 1 are processed in a reactor 2 for producing synthesis gas, for example by partial catalytic oxidation, producing a synthesis gas 3 mainly comprising H₂ and CO. The gas undergoes a Fischer-Tropsch reaction in the reactor 4 yielding liquid hydrocarbons 5 and an offgas 6. According to the invention, this offgas 6 is processed in the unit 7 in order to produce:

a gas stream 8 mainly comprising hydrogen,

a gas stream 10 mainly comprising methane, and

a gas stream 12 mainly comprising inerts (carbon dioxide, nitrogen, argon) and hydrocarbons with at least 2 carbon atoms.

Part 81 of the gas stream 8 mainly comprising hydrogen is used in a hydrocracking reactor 9, for example the one used to hydrocrack the liquid hydrocarbons 5. Another part 82 of the gas stream 8 mainly comprising hydrogen is recycled to the inlet of the Fischer-Tropsch reactor 4.

Part 101 of the gas stream 10 mainly comprising methane is recycled to the synthesis gas production reactor 2. Another part 102 is used in a steam methane reforming (SMR) reactor 11.

The stream 12 is used as fuel in a boiler 13.

By the implementation of the inventive method, the methane and hydrogen remaining in the Fischer-Tropsch process offgas can be recovered and utilized in the various reaction units of the liquid hydrocarbon production site.

The inventive method applies in particular to Fischer-Tropsch processes producing an offgas with a low CO concentration, hence to high conversion Fischer-Tropsch processes.

The method also applies to Fischer-Tropsch processes in which the recycling of the CO present in the offgas as reagent gas of the Fischer-Tropsch process is not advantageous, for example if the H₂/CO ratio of the synthesis gas upstream of the Fischer-Tropsch process is low.

### EXAMPLE

This example involves the processing of the offgas issuing from a Fischer-Tropsch process by a PSA according to the inventive method. This example is the result of the simulation of the operation of a PSA with 5 adsorbers. Each adsorber has a diameter of 0.95 metre and a height of 5 metres. The composition of an adsorber is as follows (vol %) from the top downward:
10% zeolite
10% activated carbon
70% silicalite
5% silica gel
5% alumina.

The PSA produces a stream No. 1 during its production phase. A stream No. 2 is produced during the decompression phase by intermediate withdrawal at 2.75 m from the bottom of the adsorber (or 55%), that is, in the second half of the silicalite bed. A stream No. 3 is finally produced at the end of the decompression phase of the PSA cycle.

The following results were obtained:

| | Flow Rate in Sm³/h | | | | Composition in Vol % | | | |
|---|---|---|---|---|---|---|---|---|
| | of the | of stream | of stream | of stream | of the | of stream | of stream | of stream |
| | offgas | No. 1 | No. 2 | No. 3 | offgas | No. 1 | No. 2 | No. 3 |
| H₂ | 330 | 181.5 | 26.4 | 122.1 | 33 | 95.5 | 9.5 | 23 |
| N₂ | 130 | 3.9 | 35.1 | 91 | 13 | 2.1 | 12.6 | 17.1 |
| CO | 110 | 2.2 | 30.8 | 77 | 11 | 1.2 | 11.0 | 14.5 |
| CH₄ | 240 | 2.4 | 168 | 69.6 | 24 | 1.3 | 60.2 | 13.1 |
| CO₂ | 160 | 0 | 16 | 144 | 16 | 0 | 5.7 | 27.1 |
| C₂₊ | 30 | 0 | 3 | 27 | 3 | 0 | 1.1 | 5.1 |
| Total | 1000 | 190 | 279.3 | 530.7 | | | | |

The pressure of the processed offgas was 2.1.10⁶ Pa.

The pressure of stream No. 1 was 2.10⁶ Pa.

The pressure of stream No. 2 was 3.10⁵ Pa.

The pressure of stream No. 3 was 1.5.10⁵ Pa.

It may be observed that the PSA is suitable for separating the offgas into a stream mainly comprising hydrogen, a stream mainly comprising methane and a stream mainly comprising carbon dioxide.

1-19. (canceled)

20. A method for converting hydrocarbon gases to liquid hydrocarbons using the Fischer-Tropsch process, the said Fischer-Tropsch process producing liquid hydrocarbons and an offgas comprising at least hydrogen, carbon monoxide, carbon dioxide and hydrocarbons with a maximum of 6 carbon atoms, characterized in that the offgas is subjected to a separation process producing:
at least one gas stream mainly comprising hydrogen and having a lower CO concentration than the offgas,
at least one gas stream mainly comprising methane,
at least one gas stream mainly comprising:
inerts selected from carbon dioxide, nitrogen and/or argon, and
hydrocarbons with at least 2 carbon atoms.

21. The method according to claim 1, characterized in that the gas stream mainly comprising hydrogen comprises no more than 10% of the CO present in the offgas.

22. The method according to claim 1, characterized in that the gas stream mainly comprising methane comprises at least 30% of the CO present in the offgas.

23. The method according to claim 1, characterized in that the offgas mainly comprising:
inerts selected from carbon dioxide, nitrogen and/or argon,
and hydrocarbons with at least 2 carbon atoms, comprises at least 50% of the CO present in the offgas.

24. The method according to claim 1, characterized in that the separation process uses a PSA separation unit.

25. The method according to claim 1, characterized in that each adsorber of the PSA separation unit comprises at least three adsorbent beds,
the first being composed of alumina,
the second being composed of carbon molecular sieves or silicalite,
the third being composed of activated carbon.

26. The method according to claim 1, characterized in that each adsorber of the PSA separation unit comprises a fourth bed composed of zeolite.

27. The method according to claim 25, characterized in that each adsorber of the PSA separation unit comprises a bed composed of silica gel placed between the first and the second bed.

28. The method according to claim 25, characterized in that each adsorber is divided into two semi-adsorbers in series, the first semi-adsorber comprising the alumina bed, optionally the silica gel bed, and a fraction of the carbon molecular sieve or silicalite bed.

29. The method according to claims 25, characterized in that the stream mainly comprising methane is tapped off by a withdrawal means placed in the adsorber.

30. The method according to claim 29, characterized in that the withdrawal means is placed at the level of the second half of the bed composed of carbon molecular sieves or silicalite, in the flow direction of the offgas issuing from the Fischer-Tropsch reactor in the adsorber.

31. The method according to claim 1, characterized in that at least part of the gas stream mainly comprising hydrogen is used as reagent gas in the Fischer-Tropsch process.

32. The method according to claim 1, characterized in that at least part of the gas stream mainly comprising hydrogen is used in hydrocracking processes.

33. The method according to claim 1, characterized in that at least part of the gas stream mainly comprising hydrogen is used to recover heat or work via a turbine.

34. The method according to claim 1, characterized in that at least part of the gas stream mainly comprising methane is used as reagent gas in the generation of synthesis gas.

35. The method according to claim 1, characterized in that at least part of the gas stream mainly comprising methane is used as reagent in a steam methane reforming process.

36. The method according to claim 1, characterized in that at least part of the gas stream mainly comprising inerts selected from carbon dioxide, nitrogen and/or argon, and hydrocarbons with at least 2 carbon atoms, is used as fuel.

37. The method according to claim 1, characterized in that at least part of the gas stream mainly comprising methane is used in a CO₂ separation unit.

38. The method according to claim 31, characterized in that the streams produced by the PSA separation unit are heated by heat exchange with the offgas or a synthesis gas.
